(19) United States
(12) Patent Application Publication
(10) Pub. No.: US 2010/0081991 A1
(43) Pub. Date: Apr. 1, 2010

(54) SKIN LEVEL DEVICE FOR USE WITH GASTROSTOMY TUBE

(75) Inventor: David Rork Swisher, St. Charles, MO (US)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, Mansfield, MA (US)

(21) Appl. No.: 12/545,443

(22) Filed: Aug. 21, 2009

Related U.S. Application Data
(60) Provisional application No. 61/101,231, filed on Sep. 30, 2008.

(51) Int. Cl. A61M 25/10

(52) U.S. Cl. 604/101.05

(57) ABSTRACT

A gastrostomy tube including a body having a hub portion and a tubular portion. The body defines a primary lumen having a first end in communication with an opening in the hub portion. The primary lumen has a second end in communication with an opening located in the tubular portion. The tube also includes a port supported on the body communicating with a secondary lumen. The port is adapted to engage an end of a luer tip syringe. The tube includes an external balloon located at a proximal end of the body. The tube has an internal balloon located distally from the external balloon. The internal and external balloons are in fluid communication with the port via the secondary lumen such that each is inflatable. The internal and external balloons enable a clinician to indirectly observe the internal balloon by observing the external balloon.

### CROSS-REFERENCE TO RELATED APPLICATIONS

The present application is a non-provisional of U.S. Patent Application Ser. No. 61/101,231, filed Sep. 30, 2008, and entitled, “Skin Level Device for Use with Gastrostomy Tube”, which is hereby incorporated by reference in its entirety.

### FIELD OF THE INVENTION

The present disclosure relates to gastrostomy tubes and, more particularly, to gastrostomy tubes including a skin level device having an expandable balloon and pad.

### BACKGROUND OF THE INVENTION

Low profile gastrointestinal feeding systems are frequently used for long term tube fed patients who are mobile and/or combative and who require some type of gastrostomy device to provide nutrition to their stomachs. These gastrointestinal systems may include a feeding set having a nutrition source attached to one end and a low profile gastrostomy tube connected to the other end.

The low profile gastrostomy tube is normally inserted through an established, mature stoma extending through the patient's abdominal and stomach walls. The tube is held in place by an internal retention member (e.g., an inflatable balloon or other retention means, such as a plurality of flexible retaining arms) deployed inside a patient's stomach or other visceral organ to anchor the free end of the gastrostomy tube to the organ. The internal retention member affixes a hollow organ of choice, for example, the stomach against the posterior abdominal wall of a patient. To facilitate a proper fit, some low profile gastrostomy devices use a skin level device (e.g., a skin disk) that serves as a “gap filler” on the posterior abdominal wall of a patient. The skin level device is intended to provide a tight, wiggle free connection.

Typically, a daily cleaning regimen in and around the stoma site is performed to minimize odor and the risk of infection. Unfortunately, the configuration of the skin disk sometimes requires a clinician to work around the skin disk and, in some instances, even move or remove the skin disk during the cleaning regiment. This combination of working around the skin disk and/or removing the skin disk may cause discomfort to a patient and increase the risk that the skin disk will be reassembled improperly.

As noted, gastrostomy tubes may employ an inflatable balloon that anchors the free end of the gastrostomy tube to a patient's stomach. To inflate the balloon, a clinician typically inserts a tip of a syringe into a port or valve in communication with the gastrostomy tube to inflate the balloon with a predetermined volume of fluid contained in the syringe. A clinician typically cannot see the balloon while it is being inflated because the balloon is positioned in the patient's stomach. Thus, the clinician has no visual means of knowing whether or not the balloon has actually inflated to a predetermined volume. Under-inflation and over-inflation may cause the balloon not to function as intended. For example, balloon integrity may become compromised (e.g., the balloon may dry out and loose some of its elasticity). In this instance, the balloon may rupture, over expand, or under expand during inflation, which in turn, may prevent the balloon from properly anchoring a free end of the gastrostomy tube to a stomach wall. This may prove deleterious to the patient.

### SUMMARY

In one aspect of the invention, a gastrostomy tube comprises a body having a hub portion and a tubular portion extending distally from the hub portion. The hub portion includes a top surface and a bottom surface. The body defines a primary lumen having a first end in communication with an opening in the hub portion. The primary lumen extends through the body and has a second end in communication with an opening located in a distal end of the tubular portion. The gastrostomy tube also includes a port supported on the body. The port communicates with a secondary lumen. The port is adapted to engage an end of a luer tip syringe configured for injecting fluid into the gastrostomy tube. Further, the gastrostomy tube comprises an inflatable external balloon located at a proximal end of the body. The external balloon is positioned in use to engage an outer abdominal wall of a patient. The gastrostomy tube also comprises an inflatable internal balloon located distally from the external balloon and positioned on the tubular portion to engage an interior wall of a stomach of the patient in use. The internal and external balloons are in fluid communication with the port via the secondary lumen such that each of the external and internal balloons is inflatable from a deflated condition to an inflated condition. The internal and external balloons are substantially identical to each other and configured to enable a clinician to indirectly observe the internal balloon by observing the external balloon.

In another aspect, a gastrostomy tube comprises a body having a hub portion and a tubular portion extending distally from the hub portion. The hub portion includes a top surface and a bottom surface. The body defines a primary lumen having a first end in communication with an opening in the hub portion. The primary lumen extends through the body and has a second end in communication with an opening located in a distal end of the tubular portion. The gastrostomy tube further comprises a plurality of valves supported on the body. Each of the plurality of valves communicates with a respective secondary lumen and is adapted to engage an end of a luer tip syringe configured for injecting fluid into the gastrostomy tube. In addition, the gastrostomy tube includes an inflatable external balloon located at a proximal end of the body. The external balloon is positioned in use to engage an outer abdominal wall of a patient. Further, the gastrostomy tube comprises an inflatable internal balloon located distally from the external balloon and positioned on the tubular portion to engage an interior wall of a stomach of the patient in use. Each of the internal and external balloons is in fluid communication with at least one of the plurality of valves via one of their respective secondary lumens such that each of the external and internal balloons is inflatable from a deflated condition to an inflated condition. The external balloon can be deflated while the internal balloon is inflated to facilitate cleaning of an area adjacent a stoma site of the patient.

In yet another aspect, a gastrostomy tube comprises a body having a hub portion and a tubular portion extending distally from the hub portion. The hub portion includes a top surface and a bottom surface. The body defines a primary lumen having a first end in communication with an opening in the hub portion. The primary lumen extends through the body and has a second end in communication with an opening located in a distal end of the tubular portion. The gastrostomy tube also includes a port supported on the body. The port communicates with a secondary lumen. Further, the gastrostomy tube comprises an expandable inflatable external inflatable balloon located at a proximal end of the body. The external balloon is positioned in use to engage an outer abdominal wall of a patient. The external balloon is in fluid communication with the port via the secondary lumen such that the external balloon is inflatable from a deflated condition to an inflated condition via the port. The gastrostomy tube also comprises an internal securement device located distally from the external balloon and positioned on the tubular portion to engage an interior wall of a stomach of the patient in use. The external balloon can be deflated in use while the internal securement device is in an expanded condition to facilitate cleaning of an area adjacent a stoma site of the patient.

### BRIEF DESCRIPTION OF THE DRAWINGS

Various embodiments of the presently disclosed low profile gastrostomy tube are disclosed herein with reference to the drawings wherein:

FIG. 1 is a perspective illustrating a low profile gastrostomy tube including a pair of inflatable balloons in an inflated state in accordance with a first embodiment of the present disclosure;

FIG. 2A is a side view illustrating the low profile gastrostomy tube of the first embodiment engaging an abdominal wall and a stomach wall;

FIG. 2B is a side view illustrating a low profile gastrostomy tube engaging an abdominal wall and a stomach wall in accordance with a second embodiment of the present disclosure;

FIG. 3A is a side view illustrating a low profile gastrostomy tube engaging an abdominal and stomach wall in accordance with a third embodiment of the present disclosure;

FIG. 3B is a perspective illustrating a low profile gastrostomy tube having a pair of inflatable balloons in an inflated state in accordance with a fourth embodiment of the present disclosure; and

FIG. 3C is a perspective illustrating a low profile gastrostomy tube having an inflatable balloon in an inflated state and an internal securement device having a plurality of flexible retaining arms in accordance with a fifth embodiment of the present disclosure.

### DETAILED DESCRIPTION OF EMBODIMENTS

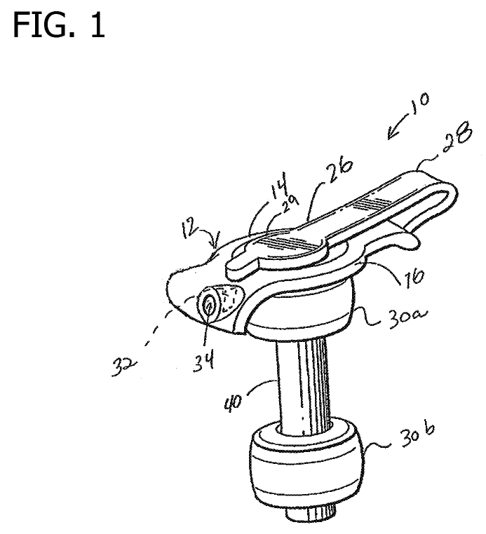

Referring to the drawings, a first embodiment of a low profile gastrostomy tube according to the present disclosure is illustrated and generally indicated as 10 in FIG. 1. The gastrostomy tube 10 may be configured for use with low profile gastrointestinal feeding systems. Known low profile gastrointestinal feeding systems suitable for use with the gastrostomy tube 10 of the present disclosure typically include a feeding set (not shown) having an elongate tube attachable to a fluid source and a connection member for securing the elongate tube to, for example, a low profile gastrostomy tube 10. A more detailed description of a feeding set suitable for use with the gastrostomy tube 10 of the present disclosure is provided in commonly owned U.S. Pat. Nos. 7,070,587 and 6,045,536 both to Meier et al., both of which are incorporated herein by reference in their entirety.

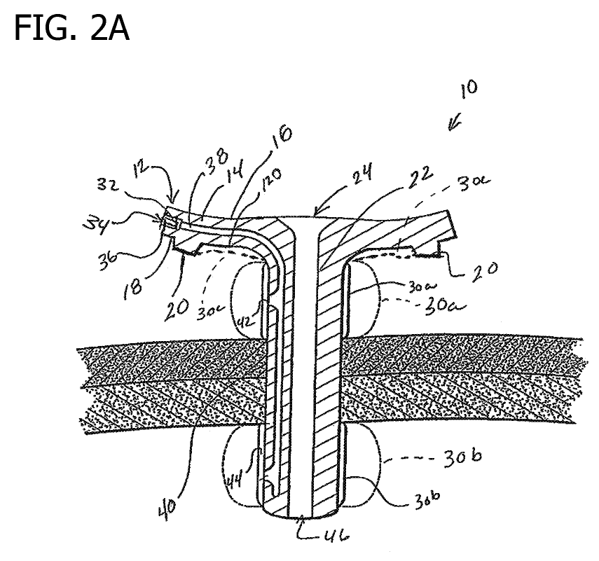

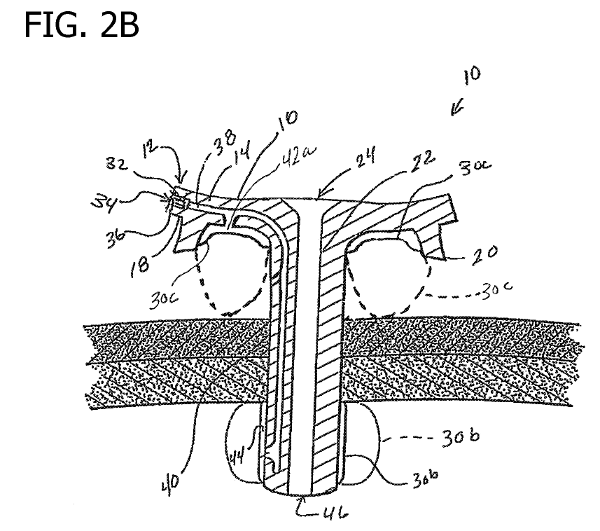

Referring to FIGS. 1 and 2A, the gastrostomy tube 10 includes a body 12 having a hub portion 14 (hub 14) and a tubular portion 40 extending distally from the hub 14. The hub 14 includes a top surface 16 and a bottom surface 18 (FIG. 2A). In some embodiments, the bottom surface 18 forms opposing legs 20 (FIG. 2A) configured and dimensioned to seat against an outer abdominal wall of a patient when a skin level device, e.g., an external inflatable balloon 30a, is in its deflated state (e.g., when cleaning a stoma). Alternatively, the bottom surface 18 of the body 12 may be substantially flat and not include legs 20. In the drawings, the size of opposing legs 20 has been exaggerated for illustrative purposes.

The gastrostomy tube 10 includes a primary lumen 22 extending through the body 12 in communication with a proximal opening 24. As shown in FIG. 1, a tethered cap 26 is affixed to or integrally formed with the hub 14 (FIG. 1). The cap 26 includes a tether 28 attaching a cap member 29 to the hub 14 (FIG. 1). The cap member 29 may be configured to seal the primary lumen 22. An annular undercut may be formed around the circumference of the primary lumen 22 near the opening 24 of the lumen for engaging an annular flange (not shown) of the cap member 29 to secure the cap member in the primary lumen 22. Engagement between the annular flange and the undercut is preferably a snap fit engagement. Alternatively, an interference fit or a combination of interference and snap fit engagement may be used for the same purpose. The hub 14 may also include a valve member (not shown) positioned across the primary lumen 22 for sealing off the primary lumen to fluid flow. For a more detailed description of the aforementioned components, parts, and/or members associated with the low profile gastrostomy tube 10, reference may be made to commonly owned U.S. Pat. No. 7,070,587.

The hub 14 includes one or more ports having one or more valves 32 (hereinafter collectively referred to as valves 32 and shown in phantom in FIG. 1 and schematically in FIG. 2A) extending longitudinally from the body 12. The valve 32 includes an opening 34 that communicates with a passage 36 (FIG. 2A) and is adapted to engage an end of a luer tip syringe (not shown) or other suitable device for injecting fluid into the gastrostomy tube 10 to inflate one or more of the inflatable balloons as shall be discussed in greater detail below. The valve member 32 is positioned across the passage 36 for providing a fluid tight barrier allowing fluid to enter the valve when the luer tip syringe is properly engaged to it. However, when the end of the luer tip syringe is disengaged from the valve 32, the valve portion reseals itself, preventing fluid from escaping from the valve. Such valves are known in the art and will not be described in further detail.

With reference to FIG. 2A, the passage 36 communicates with a secondary lumen 38 extending through the hub 14 and communicating with an external balloon 30a and an internal balloon 30b through a tubular portion 40 of the gastrostomy tube 10. The secondary lumen 38 extends axially through the tubular portion 40 and terminates within interiors 42, 44 of the balloons 30a, 30b, respectively, attached to or integral with the tubular portion 40.

As illustrated in FIG. 2A, both the primary lumen 22 and the secondary lumen 38 extend axially through the tubular portion 40 with the primary lumen terminating at a distal opening 46 and the secondary lumen communicating with the interiors 42, 44 of the inflatable balloons 30a, 30b, respectively. The secondary lumen 38 provides a fluid conduit between the valve 32 and the inflatable balloons 30a, 30b to effect inflation/deflation of the balloons and to facilitate securing and removing the gastrostomy tube 10 with respect to the stoma site.

The external inflatable balloon 30a is configured for seating the gastrostomy tube 10 on the outer abdominal wall of a patient and acting as a “gap filler” between the bottom surface 18 of the hub 14 and the outer abdominal wall of a patient to obtain a tight, wiggle free connection between the gastrostomy tube and the patient. In the drawings, the height of the low profile gastrostomy tube 10 relative to an outer abdominal wall of a patient has been exaggerated for illustrative purposes. The external balloon 30a may also act as a secondary seal at the stoma site. With this purpose in mind, the external inflatable balloon 30a is located on the proximal end of the tubular member 40 adjacent the hub 14. Alternatively, instead of the balloon 30a, an external balloon 30c may be operatively disposed on the bottom surface 18 of the hub 14 (as shown in phantom in FIG. 2A). In this instance, the secondary lumen 38 would be configured to communicate with an interior 42a of the balloon 30c so the balloon 30c could be expanded to perform the above-described functions of the balloon 30a (see FIG. 2B, for example).

The internal inflatable balloon 30b is configured for affixing a hollow organ, i.e., the stomach, against the posterior abdominal wall of the patient. Each of the inflatable balloons 30a, 30b, or 30c is constructed from an elastomeric or other flexible material of similar thickness permitting the inflatable balloons 30a, 30b to assume an inflated state when fluid is injected into their respective interiors 42, 44 through the secondary lumen 38.

In use, the gastrostomy tube 10 is inserted through a stoma on a patient (FIG. 2A) while the balloons 30a, 30b are in a deflated state. To inflate the balloons 30a, 30b, a clinician engages the tip of a syringe to the valve 32 and injects fluid into the passage 36 and through the secondary lumen 38. As fluid travels through the secondary lumen 38 it enters the interiors 42, 44 of the balloons 30a, 30b (or 30c) and inflates the respective balloons 30a, 30b (shown in phantom in FIGS. 2A and 2B, for example). As noted above, the height of the low profile gastrostomy tube 10 relative to an outer abdominal wall of a patient when the external balloons 30a or 30c are inflated has been exaggerated for illustrative purposes. Once the balloons 30a, 30b are inflated, the gastrostomy tube 10 is intended to remain comfortably and securely connected to a patient so the primary lumen 22 may act as a conduit for delivering fluid directly into the patient's stomach or other visceral organ. To deflate the balloons 30a, 30b (e.g., to clean in and/or around the stoma), the clinician withdraws fluid from the interiors 42, 44 and through the valve 32 by pulling back on the plunger of a syringe until all the fluid has been fully evacuated from the balloons 30a, 30b. Because the internal balloon 30a and the external balloon 30b are substantially identical and formed from the same material, have the same thicknesses and are interconnected by a single lumen 38, the volume of the internal balloon 30b will correspond to the volume of the external balloon 30a. Thus, the clinician may observe the external balloon 30a to obtain an estimate of the size of the internal balloon 3b. This configuration aides the clinician in accurately inflating the internal balloon 30b.

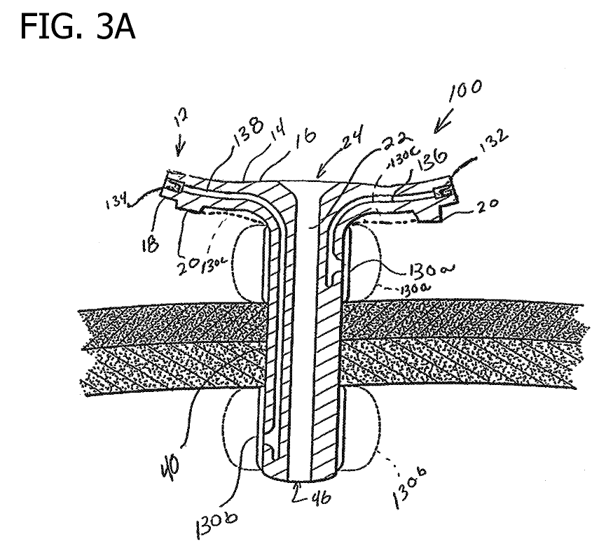

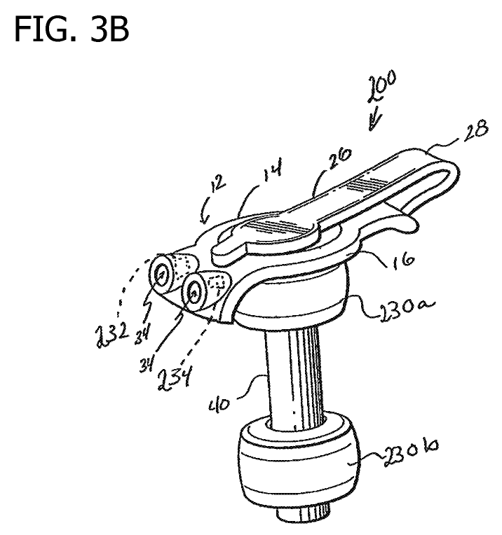

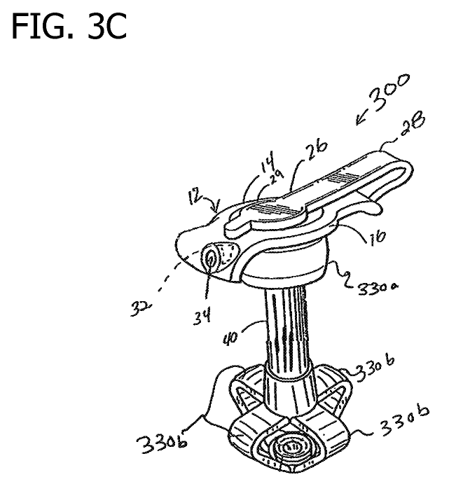

Referring now to FIG. 3A, another embodiment of a low profile gastrostomy tube according to the present disclosure is illustrated and generally indicated as 100. In the embodiment illustrated in FIG. 3A, a pair of valves 132, 134 (shown schematically) are positioned on opposite sides of the hub 14. The operative features of the gastrostomy tube 100 are substantially similar to the gastrostomy tube 10. However, in the embodiment illustrated in FIG. 3A, the gastrostomy tube 100 includes two valves 132, 134 that independently communicate with the balloons 130a (or 130c) and 130b, respectively, by way of respective secondary lumens 136, 138. In this instance, each of the balloons 130a (or 130c) and 130b is independently inflatable and deflatable by way of their respective valves 132, 134. This combination of valves 132, 134 and balloons 130a (or 130c) and 130b facilitates cleaning in and around the stoma site, because the external balloon can be deflated without deflating the internal balloon. Deflating the external balloon 130a allows for greater access to the stoma site to enable a clinician to clean around the stoma site while maintaining the gastrostomy tube 100 secured to a patient.

Referring now to FIG. 3B, another alternate embodiment of a low profile gastrostomy tube according to the present disclosure is illustrated and generally indicated as 200. In the embodiment illustrated in FIG. 3B, the gastrostomy tube 200 is shown having two valves 232, 234 (shown in phantom) configured to independently communicate with the balloons 230a, 230b. In this instance, the valves 232, 234 are positioned on the same side of the hub 14 and adjacent one another. The operative features of gastrostomy tube 200 are substantially similar to gastrostomy tube 100. As with each of the balloons 130a, 130b described above with respect to the previous embodiment, each of the balloons 230a, 230b of this embodiment are independently inflatable and deflatable by way of their respective valves 232, 234.

While the above-described gastrostomy tubes (e.g., gastrostomy tube 10) of the present disclosure have been described in terms of use with an internal balloon member (e.g., 30b) as a means for securing the gastrostomy tube inside a visceral organ of a patient, other means for securing the tube are contemplated.

For example, referring now to FIG. 3C, yet another alternate embodiment of a low profile gastrostomy tube according to the present disclosure is illustrated and generally indicated as 300. In the embodiment illustrated in FIG. 3C, the gastrostomy tube 300 is shown having an internal securement device including a cage having one or more flexible arms 330b (four flexible arms are shown). The flexible arms 330b are movable from a collapsed configuration enabling a clinician to insert the gastrostomy tube 300 through an established, mature stoma of a patient to a expanded configuration (FIG. 3C) enabling a clinician to anchor or secure the stomach against the posterior abdominal wall of a patient. For a more detailed description of the operative features of the flexible arms 330b reference can be again made to commonly owned U.S. Pat. No. 7,070,587. In the embodiment illustrated in FIG. 3C, a secondary lumen (see FIG. 2A for example) would be configured to communicate with an interior 42 of the balloon 330a so the balloon can be expanded to perform the above-described functions of the balloon 30a.

It will be understood that various modifications may be made to the embodiments disclosed herein. Therefore, the above description should not be construed as limiting, but merely as examples of preferred embodiments. Those skilled in the art will envision other modifications within the scope and spirit of the claims appended hereto.

When introducing elements of the present invention or the preferred embodiments(s) thereof, the articles “a”, “an”, “the”, and “said” are intended to mean that there are one or more of the elements. The terms “comprising”, “including”, and “having” are intended to be inclusive and mean that there may be additional elements other than the listed elements.

What is claimed is:

1. A gastrostomy tube comprising:
a body having a hub portion and a tubular portion extending distally from the hub portion, the hub portion including a top surface and a bottom surface, the body defining a primary lumen having a first end in communication with an opening in the hub portion, the primary lumen extending through the body and having a second end in communication with an opening located in a distal end of the tubular portion;
a port supported on the body, the port communicating with a secondary lumen, the port being adapted to engage an end of a luer tip syringe configured for injecting fluid into the gastrostomy tube;
an inflatable external balloon located at a proximal end of the body, the external balloon being positioned in use to engage an outer abdominal wall of a patient; and
an inflatable internal balloon located distally from the external balloon and positioned on the tubular portion to engage an interior wall of a stomach of the patient in use, the internal and external balloons being in fluid communication with the port via the secondary lumen such that each of the external and internal balloons is inflatable from a deflated condition to an inflated condition;
wherein the internal and external balloons are substantially identical to each other and configured to enable a clinician to indirectly observe the internal balloon by observing the external balloon.

2. The gastrostomy tube according to claim 1, wherein the gastrostomy tube is configured for use with a low profile gastrointestinal feeding system.

3. The gastrostomy tube according to claim 1, wherein the hub portion includes a bottom surface forming opposing legs configured and dimensioned to seat against the outer abdominal wall of a patient is use.

4. The gastrostomy tube according to claim 1, wherein the external balloon is configured to provide a secondary seal between the outer abdominal wall and an interior wall of a stomach of a patient in use.

5. The gastrostomy tube according to claim 1, wherein the external balloon is operatively disposed on the bottom surface of the hub portion and in fluid communication with a valve via the secondary lumen such that each of the external and internal balloons is inflatable from a deflated condition to an inflated condition.

6. The gastrostomy tube according to claim 1, wherein the hub portion includes a tethered cap which includes a tether attaching a cap member to the opening of the hub portion.

7. A gastrostomy tube comprising:
a body having a hub portion and a tubular portion extending distally from the hub portion, the hub portion including a top surface and a bottom surface, the body defining a primary lumen having a first end in communication with an opening in the hub portion, the primary lumen extending through the body and having a second end in communication with an opening located in a distal end of the tubular portion;
a plurality of valves supported on the body, each of the plurality of valves communicating with a respective secondary lumen and being adapted to engage an end of a luer tip syringe configured for injecting fluid into the gastrostomy tube;
an inflatable external balloon located at a proximal end of the body, the external balloon being positioned in use to engage an outer abdominal wall of a patient; and
an inflatable internal balloon located distally from the external balloon and positioned on the tubular portion to engage an interior wall of a stomach of the patient in use, each of the internal and external balloons being in fluid communication with at least one of the plurality of valves via one of their respective secondary lumens such that each of the external and internal balloons is inflatable from a deflated condition to an inflated condition;
wherein the external balloon can be deflated while the internal balloon is inflated to facilitate cleaning of an area adjacent a stoma site of the patient.

8. The gastrostomy tube according to claim 7, wherein each of the plurality of valves is operatively disposed on opposite sides of the hub portion.

9. The gastrostomy tube according to claim 7, wherein each of the plurality of valves is positioned on a same side of the hub portion.

10. The gastrostomy tube according to claim 7, wherein the external balloon is configured to provide a secondary seal between an outer abdominal wall and an interior wall of a stomach of a patient in use.

11. The gastrostomy tube according to claim 7, wherein the external balloon is operatively disposed on the bottom surface of the hub portion and in fluid communication with at least one of the plurality valves via one of the respective secondary lumens such that the external balloon is inflatable from a deflated condition to an inflated condition.

12. The gastrostomy tube according to claim 7, wherein the gastrostomy tube is configured for use with a low profile gastrointestinal feeding system.

13. The gastrostomy tube according to claim 7, wherein the hub portion includes a bottom surface forming opposing legs configured and dimensioned to seat against an outer abdominal wall of a patient in use.

14. The gastrostomy tube according to claim 7, wherein the external balloon is configured to provide a secondary seal between an outer abdominal wall and an interior wall of a stomach of a patient in use.

15. The gastrostomy tube according to claim 7, wherein the hub portion includes a tethered cap having a tether attaching a cap member to the opening of the hub portion.

16. A gastrostomy tube comprising:
a body having a hub portion and a tubular portion extending distally from the hub portion, the hub portion including a top surface and a bottom surface, the body defining a primary lumen having a first end in communication with an opening in the hub portion, the primary lumen extending through the body and having a second end in communication with an opening located in a distal end of the tubular portion;
a port supported on the body, the port communicating with a secondary lumen;
an inflatable external inflatable balloon located at a proximal end of the body, the external balloon being positioned in use to engage an outer abdominal wall of a patient, the external balloon being in fluid communication with the port via the secondary lumen such that the external balloon is inflatable from a deflated condition to an inflated condition via the port; and
an expandable internal securement device located distally from the external balloon and positioned on the tubular portion to engage an interior wall of a stomach of the patient in use;
wherein the external balloon can be deflated in use while the internal securement device is in an expanded condition to facilitate cleaning of an area adjacent a stoma site of the patient.

17. The gastrostomy tube according to claim 16, wherein the internal securement device includes a plurality of flexible arms.
